(19) United States
(12) Patent Application Publication
(10) Pub. No.: US 2026/0144636 A1
(43) Pub. Date: May 28, 2026

(54) DELIVERY DEVICE FOR A REPLACEMENT HEART VALVE IMPLANT

(71) Applicant: BOSTON SCIENTIFIC SCIMED, INC, MAPLE GROVE, MN (US)

(72) Inventors: Jessica D. Worlock, Hopkins, MN (US); Levi Joel Wolterstorff, Saint Paul, MN (US); Adam Troy Olson, Plymouth, MN (US); Philip Andrew Litecky, Forest Lake, MN (US); Kristi Mae Flury, Maple Grove, MN (US); Stacey Lynn Tonne, Burnsville, MN (US)

(73) Assignee: BOSTON SCIENTIFIC SCIMED, INC., MAPLE GROVE, MN (US)

(21) Appl. No.: 19/398,859

(22) Filed: Nov. 24, 2025

Related U.S. Application Data
(60) Provisional application No. 63/724,567, filed on Nov. 25, 2024.

(51) Int. Cl. A61F 2/24

(52) U.S. Cl. CPC A61F 2/2436

(57) ABSTRACT

A delivery device for a replacement heart valve implant includes a handle having first and second rotatable knobs, and an elongate shaft assembly extending from the handle. A distal portion of the shaft assembly includes an implant holding portion configured to constrain the implant in a radially collapsed configuration. The implant includes a first feature and a second feature. The first knob includes a first tactile element representative of the first feature. The second knob includes a second tactile element representative of the second feature. The first tactile element may be embossed on the first knob. The second tactile element may be embossed on the second knob. The first and second tactile elements are configured to reduce a user's cognitive load during deployment of the implant.

### CROSS-REFERENCE TO RELATED APPLICATIONS

This application claims the benefit of priority of U.S. Provisional Application No. 63/724,567 filed Nov. 25, 2024, the entire disclosure of which is hereby incorporated by reference.

### TECHNICAL FIELD

The disclosure relates generally to medical devices and more particularly to medical devices that are adapted for implanting stents and medical devices including a stent component.

### BACKGROUND

A wide variety of intracorporeal medical devices and/or implants have been developed for medical use including artificial heart valve implants for repair or replacement of diseased heart valves. Some delivery devices may require a number of steps in a particular order to properly deploy an implant. Some users may perform a low volume of cases, and familiarity with the delivery device may be limited. Confusion, cognitive load, and/or stress may slow and/or complicate the procedure. Of the known medical devices, systems, and methods, each has certain advantages and disadvantages. There is an ongoing need to provide alternative devices, systems, and methods for loading medical devices and/or heart valve implants into a delivery system.

### SUMMARY

In one example, a delivery device for a replacement heart valve implant may comprise a handle comprising a first rotatable knob and a second rotatable knob, and an elongate shaft assembly extending distally from the handle, wherein a distal portion of the elongate shaft assembly comprises an implant holding portion configured to constrain the replacement heart valve implant in a radially collapsed configuration. The replacement heart valve implant may comprise a first feature and a second feature. The first rotatable knob may comprise a first tactile element representative of the first feature. The second rotatable knob may comprise a second tactile element representative of the second feature.

In addition, or alternatively, to any example described herein, the first tactile element has a first shape corresponding to a shape of the first feature.

In addition, or alternatively, to any example described herein, the second tactile element has a second shape corresponding to a shape of the second feature.

In addition, or alternatively, to any example described herein, rotation of the first rotatable knob relative to the handle is configured to deploy the first feature from the implant holding portion.

In addition, or alternatively, to any example described herein, rotation of the second rotatable knob relative to the handle is configured to deploy the second feature from the implant holding portion.

In addition, or alternatively, to any example described herein, the first rotatable knob has a first axial length, and the second rotatable knob has a second axial length greater than the first axial length.

In addition, or alternatively, to any example described herein, the first rotatable knob is formed from a different material than the handle.

In addition, or alternatively, to any example described herein, the second rotatable knob is formed from a different material than the handle.

In addition, or alternatively, to any example described herein, the first rotatable knob comprises an outer surface and the first tactile element extends radially outward of the outer surface of the first rotatable knob.

In addition, or alternatively, to any example described herein, the second rotatable knob comprises an outer surface and the second tactile element extends radially outward of the outer surface of the second rotatable knob.

In addition, or alternatively, to any example described herein, the first tactile element is monolithically formed with the first rotatable knob.

In addition, or alternatively, to any example described herein, the second tactile element is monolithically formed with the second rotatable knob.

In addition, or alternatively, to any example described herein, and in a second example, a delivery device for a replacement heart valve implant may comprise a handle comprising a first rotatable knob and a second rotatable knob, and an elongate shaft assembly extending distally from the handle, wherein a distal portion of the elongate shaft assembly comprises an implant holding portion configured to constrain the replacement heart valve implant in a radially collapsed configuration. The replacement heart valve implant may comprise a first feature and a second feature. A first tactile element representative of the first feature may be embossed on the first rotatable knob. A second tactile element representative of the second feature may be embossed on the second rotatable knob.

In addition, or alternatively, to any example described herein, the handle comprises an ergonomic outer surface extending from the first rotatable knob to a distal end of the handle.

In addition, or alternatively, to any example described herein, the delivery device may comprise a safety pin configured to engage with the handle to prevent rotation of the second rotatable knob relative to the handle.

In addition, or alternatively, to any example described herein, the safety pin is configured to extend through the second rotatable knob to engage with the handle.

In addition, or alternatively, to any example described herein, the handle may comprise a landmark ring disposed proximate a distal end of the second rotatable knob. The landmark ring may comprise a plurality of distally facing directional markings configured to aid in loading the replacement heart valve implant into the implant holding portion.

In addition, or alternatively, to any example described herein, the first rotatable knob may comprise a plurality of distally facing directional markings configured to aid in loading the replacement heart valve implant into the implant holding portion.

In addition, or alternatively, to any example described herein, the landmark ring is fixedly attachable to the second rotatable knob.

In addition, or alternatively, to any example described herein, and in a third example, a delivery device for a replacement heart valve implant may comprise a handle comprising a first rotatable knob and a second rotatable knob, and an elongate shaft assembly extending distally from the handle, wherein a distal portion of the elongate shaft assembly comprises an implant holding portion configured to constrain the replacement heart valve implant in a radially collapsed configuration. The first rotatable knob and the second rotatable knob may comprise tactile elements configured to reduce a user's cognitive load during deployment of the replacement heart valve implant from the implant holding portion.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 schematically illustrate selected aspects of a replacement heart valve implant;

FIG. 3 schematically illustrates selected aspects of a replacement heart valve system including the replacement heart valve implant and a delivery device;

FIG. 4 schematically illustrates selected aspects of a handle of the delivery device; and

FIGS. 5-6 schematically illustrate selected aspects related to deploying the replacement heart valve implant from the delivery device.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently - such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

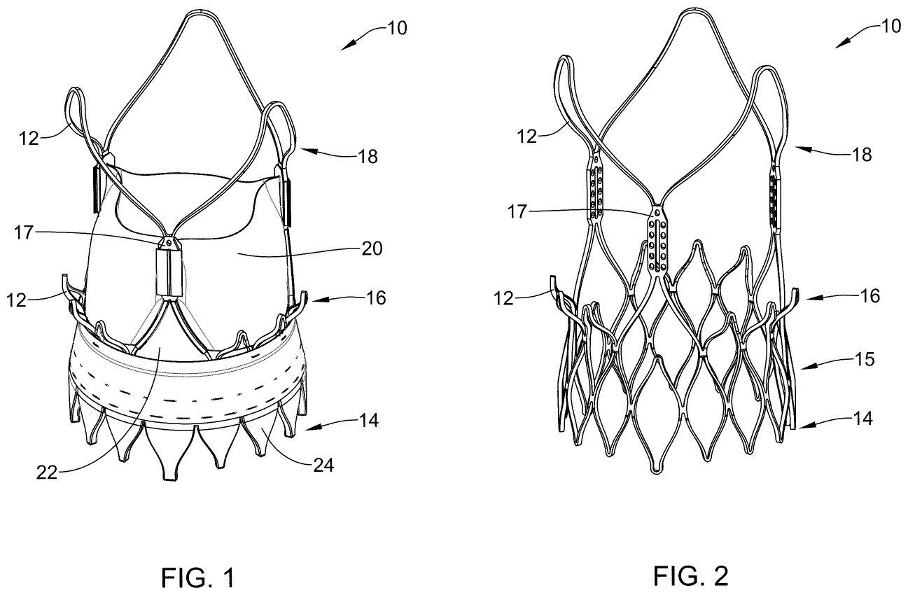

FIGS. 1-2 illustrate selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

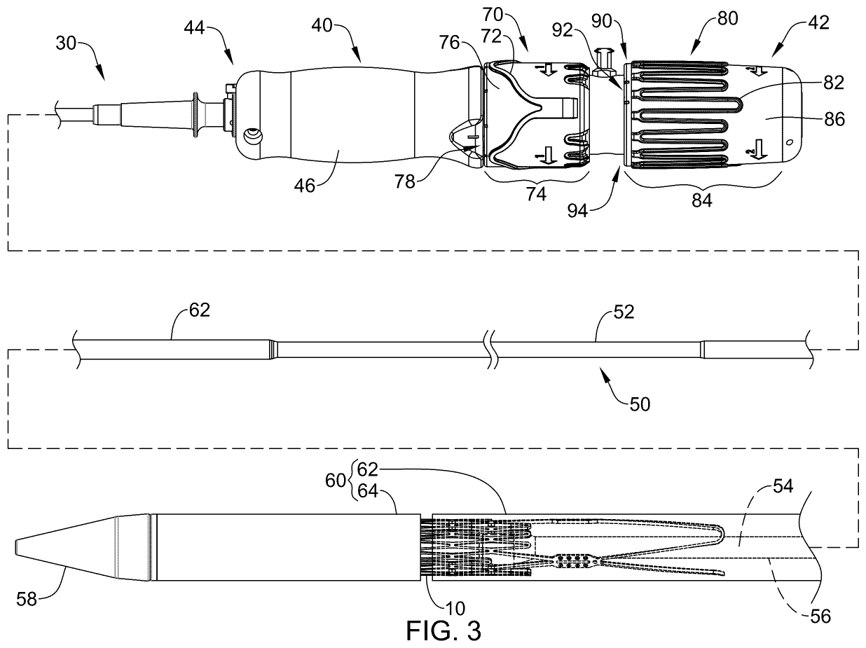

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The replacement heart valve implant 10 and/or the expandable framework 12 may be configured to shift between a radially collapsed configuration (e.g., FIG. 3) and a radially expanded configuration (e.g., FIGS. 1-2). In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations, including combinations thereof, are also contemplated.

FIG. 2 illustrates some aspects of the expandable framework 12 in more detail. In some embodiments, the expandable framework 12 may define a lower crown 14 proximate and/or at an inflow end, an upper crown 16 proximate and/or at an outflow end, and a plurality of stabilization arches 18 extending downstream from the outflow end. The expandable framework may comprise a body portion 15 extending between the lower crown 14 and the upper crown 16. In at least some embodiments, the body portion 15 may be formed as and/or similar to a stent or a stent-like structure. In some embodiments, the body portion 15 may comprise a plurality of closed cells. In some embodiments, the expandable framework 12 may comprise a plurality of commissure posts 17. In some embodiments, the plurality of commissure posts 17 may extend downstream of and/or away from the upper crown 16. In some embodiments, the plurality of stabilization arches 18 may extend downstream of and/or away from the upper crown 16 and/or the plurality of commissure posts 17 in a direction opposite the lower crown 14. In some embodiments, the upper crown 16 and/or the plurality of commissure posts 17 may be disposed longitudinally and/or axially between the lower crown 14 and the plurality of stabilization arches 18.

In some embodiments, the replacement heart valve implant 10 and/or the expandable framework 12 may include a proximal portion and a distal portion. In some embodiments, orientation of the replacement heart valve implant 10 may be related to a delivery device 30 (e.g., FIG. 3) and/or a direction of implantation relative to a target site (e.g., a native heart valve). In some embodiments, the proximal portion may include the outflow end and/or the plurality of stabilization arches 18. In some embodiments, the proximal portion may include the upper crown 16 and/or the plurality of commissure posts 17. In some embodiments, the distal portion may include the inflow end and/or the lower crown 14. In some embodiments, the distal portion may include the body portion 15 and/or a portion of the body portion 15 disposed immediately adjacent the lower crown 14. Other configurations are also contemplated.

Returning to FIG. 1, in some embodiments, the replacement heart valve implant 10 may include a plurality of valve leaflets 20 disposed within the central lumen. The plurality of valve leaflets 20 may be coupled, secured, and/or fixedly attached to the expandable framework 12 at the plurality of commissure posts 17 to form and/or define a plurality of commissures. In addition, or alternatively, in some embodiments, the plurality of valve leaflets 20 may be coupled, secured, and/or fixedly attached to the expandable framework 12 proximate and/or at other locations, such as the inflow end, the lower crown 14, etc. The plurality of valve leaflets 20 may be configured to shift between an open position and a closed position. The plurality of valve leaflets 20 may be configured to substantially restrict fluid flow through the replacement heart valve implant 10 in the closed position. The plurality of valve leaflets 20 may move apart from each other and/or radially outward within the central lumen in the open position to permit fluid flow through the replacement heart valve implant 10 and/or the central lumen.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along an outer surface of the expandable framework 12. In some embodiments, the outer skirt 24 may be disposed at and/or adjacent the lower crown 14. The outer skirt 24 may ensure the fluid flows through the replacement heart valve implant 10 and does not flow around the replacement heart valve implant 10 (e.g., between the expandable framework 12 and the vessel wall).

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of a plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 17 mm (mm) (0.669 inches), about 19.5 mm (0.768 inches), about 23 millimeters (0.905 inches), about 25 mm (0.984 inches), about 27 mm (1.063 inches), about 30 mm (1.181 inches), about 31 mm (1.220 inches), etc. in an unconstrained configuration (e.g., in the radially expanded configuration). In some embodiments, the expandable framework 12 and/or the replacement heart valve implant 10 may have an outer extent of about 11 mm (0.433 inches), about 10 mm (0.394 inches), about 9 mm (0.354 inches), about 8 mm (0.315 inches), about 7 mm (0.276 inches), about 6 mm (0.236 inches), about 5 mm (0.197 inches), about 4 mm (0.157 inches), etc. in the radially collapsed configuration. Other configurations are also contemplated.

In some embodiments, the replacement heart valve implant 10 and/or the expandable framework 12 may comprise a first feature and a second feature. In some embodiments, the first feature may be and/or may correspond to the proximal portion of the replacement heart valve implant 10 and/or the expandable framework 12. In some embodiments, the first feature may comprise the plurality of stabilization arches 18, the plurality of commissure posts 17, and/or the upper crown 16. In some embodiments, the second feature may be and/or may correspond to the distal portion of the replacement heart valve implant 10 and/or the expandable framework 12. In some embodiments, the second feature may comprise the lower crown 14, the body portion 15, and/or the portion of the body portion 15 disposed immediately adjacent the lower crown 14. Other configurations are also contemplated.

In some embodiments, the first feature and/or the second feature of the replacement heart valve implant 10 and/or the expandable framework 12 may comprise and/or include one or more elements (or portions thereof) of the replacement heart valve implant 10 coupled, secured, and/or fixedly attached to the first feature and/or the second feature. In some embodiments, the first feature may comprise and/or include the plurality of valve leaflets 20 or portions thereof. In some embodiments, the second feature may comprise and/or include the plurality of valve leaflets 20 or portions thereof, the inner skirt 22, and/or the outer skirt 24. Other configurations are also contemplated.

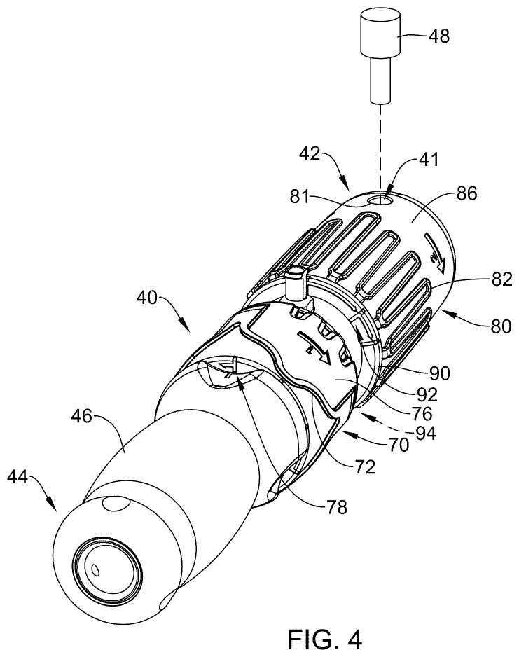

FIGS. 3-4 illustrate selected aspects of a replacement heart valve system comprising the replacement heart valve implant 10 and a delivery device 30 for delivering the replacement heart valve implant 10 to a native heart valve. It should be noted that FIG. 3 includes at least one change of scale (e.g., all parts of the figure are not drawn to the same scale) to improve viewability and show additional detail of selected aspects of the delivery device 30. Additionally, the expandable framework 12 is shown in FIG. 3 in the radially collapsed configuration and some elements of the replacement heart valve implant 10 are omitted to improve clarity. It should further be noted that all elements are not shown or labelled in each figure in the interest of clarity.

The delivery device 30 may include a handle 40 and an elongate shaft assembly 50 extending distally from the handle 40. The handle 40 may include a proximal end 42 and a distal end 44 opposite the proximal end 42. The elongate shaft assembly 50 may extend distally from the distal end 44 of the handle 40. Some aspects of the handle 40 are shown in more detail in FIG. 4. In some embodiments, the handle 40 may include one or more rotatable knobs. In some embodiments, the one or more rotatable knobs may include a first rotatable knob 70 and a second rotatable knob 80. In at least some embodiments, the first rotatable knob 70 and/or the second rotatable knob 80 may be configured to rotate around a central longitudinal axis of the delivery device 30 and/or the handle 40. Other configurations and/or types of interface controls are also contemplated. For example, the handle 40 may comprise one or more sliders, buttons, switches, touch-sensitive elements, etc. in place of, in addition to, or in combination with the one or more rotatable knobs. In some embodiments, the handle 40 may comprise an ergonomic outer surface 46 extending from the first rotatable knob 70 toward and/or to the distal end 44 of the handle 40. In some embodiments, the ergonomic outer surface 46 of the handle 40 may be formed and/or shaped to facilitate easy grasping and/or gripping by a user's hand. In some embodiments, the ergonomic outer surface 46 of the handle 40 may be smooth. In some embodiments, the ergonomic outer surface 46 of the handle 40 may be textured. Other configurations, including combinations thereof, are also contemplated.

In biomechanics, two technically understood hand grips are the power grip and the pinch grip (or precision grip). In some embodiments, a pinch grip may be configured for slow and/or precise movements using the fingers and thumb (e.g., fine motor control), while a power grip may be configured for fast and/or gross movements using the entire hand. In some embodiments, a power grip may require and/or facilitate the transfer of more strength than a pinch grip, and/or may permit and/or facilitate faster movement than a pinch grip. In some embodiments, the ergonomic outer surface 46 of the handle 40 may be configured for and/or to provide a power grip (for a user's left hand, for example). In some embodiments, the first rotatable knob 70 may be configured for and/or to provide a pinch grip (for the user's right hand, for example). In some embodiments, the second rotatable knob 80 may be configured for and/or to provide a power grip (for the user's right hand, for example). Other configurations are also contemplated.

In some embodiments, the first rotatable knob 70 may comprise a first tactile element 72 representative of and/or corresponding to the first feature of the replacement heart valve implant 10 and/or the expandable framework 12. In some embodiments, the first tactile element 72 may have a first shape and/or a first form corresponding to a shape of the first feature of the replacement heart valve implant 10 and/or the expandable framework 12. In some embodiments, the first rotatable knob 70 may comprise an outer surface 76. In some embodiments, the first tactile element 72 may extend radially outward of the outer surface 76 of the first rotatable knob 70 (e.g., the first tactile element 72 may be raised from and/or relative to the outer surface 76). In some alternative embodiments, the first tactile element 72 may extend radially inward from the outer surface 76 of the first rotatable knob 70 (e.g., the first tactile element 72 may be recessed from and/or relative to the outer surface 76). In at least some embodiments, the first tactile element 72 may be monolithically formed with the first rotatable knob 70. In some embodiments, the first tactile element 72 may be formed from a different material than the first rotatable knob 70. In some embodiments, the first tactile element 72 representative of the first feature may be embossed on the first rotatable knob 70. In some embodiments, the first tactile element 72 representative of the first feature may be overlaid on the outer surface 76 of the first rotatable knob 70. In some embodiments, the first tactile element 72 may be fixedly attached to the outer surface 76 of the first rotatable knob 70. In some embodiments, the first tactile element 72 representative of the first feature may aid in distinguishing the first rotatable knob 70 from the handle 40. Other configurations are also contemplated.

In some embodiments, the second rotatable knob 80 may comprise a second tactile element 82 representative of and/or corresponding to the second feature of the replacement heart valve implant 10 and/or the expandable framework 12. In some embodiments, the second tactile element 82 may have a second shape and/or a second form corresponding to a shape of the second feature of the replacement heart valve implant 10 and/or the expandable framework 12. In some embodiments, the second rotatable knob 80 may comprise an outer surface 86. In some embodiments, the second tactile element 82 may extend radially outward of the outer surface 86 of the second rotatable knob 80 (e.g., the second tactile element 82 may be raised from and/or relative to the outer surface 86). In some alternative embodiments, the second tactile element 82 may extend radially inward from the outer surface 86 of the second rotatable knob 80 (e.g., the second tactile element 82 may be recessed from and/or relative to the outer surface 86). In at least some embodiments, the second tactile element 82 may be monolithically formed with the second rotatable knob 80. In some embodiments, the second tactile element 82 may be formed from a different material than the second rotatable knob 80. In some embodiments, the second tactile element 82 representative of the second feature may be embossed on the second rotatable knob 80. In some embodiments, the second tactile element 82 representative of the second feature may be overlaid on the outer surface 86 of the second rotatable knob 80. In some embodiments, the second tactile element 82 may be fixedly attached to the outer surface 86 of the second rotatable knob 80. In some embodiments, the second tactile element 82 representative of the second feature may aid in distinguishing the second rotatable knob 80 from the handle 40 and/or from the first rotatable knob 70. Other configurations are also contemplated.

In some embodiments, the first rotatable knob 70 may have a first axial length 74 (e.g., FIG. 3), and the second rotatable knob 80 may have a second axial length 84 (e.g., FIG. 3) greater than the first axial length 74. Other configurations are also contemplated. In some embodiments, the first rotatable knob 70 and/or the second rotatable knob 80 may be formed from a different material than the handle 40. In some embodiments, the first rotatable knob 70 and/or the second rotatable knob 80 may be formed from the same material, which may be different from the handle 40. In some embodiments, the first rotatable knob 70 and/or the second rotatable knob 80 may be formed from different materials, which are both different from the handle 40. Other configurations are also contemplated.

In some embodiments, the first rotatable knob 70 (and/or elements thereof, which may include but are not limited to the first tactile element 72 and/or the outer surface 76) may have a first coefficient of friction greater than a coefficient of friction of the ergonomic outer surface 46 of the handle 40. In some embodiments, the second rotatable knob 80 (and/or elements thereof, which may include but are not limited to the second tactile element 82 and/or the outer surface 86) may have a second coefficient of friction greater than the coefficient of friction of the ergonomic outer surface 46 of the handle 40. In some embodiments, the first rotatable knob 70 (and/or elements thereof, which may include but are not limited to the first tactile element 72 and/or the outer surface 76) and/or the second rotatable knob 80 (and/or elements thereof, which may include but are not limited to the second tactile element 82 and/or the outer surface 86) may be textured to improve a user's grip thereon and/or to further distinguish the first rotatable knob 70 and/or the second rotatable knob 80 from the handle 40. Other configurations are also contemplated.

In some embodiments, the first rotatable knob 70 and the second rotatable knob 80 may be rotatable around and/or relative to the handle 40. As discussed herein, rotation of the first rotatable knob 70 around and/or relative to the handle 40 may be configured to deploy the first feature and/or the proximal portion of the replacement heart valve implant 10 from an implant holding portion 60 of the delivery device 30. Similarly, rotation of the second rotatable knob 80 around and/or relative to the handle 40 may be configured to deploy the second feature and/or the distal portion of the replacement heart valve implant 10 from the implant holding portion 60 of the delivery device 30.

In some embodiments, the first rotatable knob 70 and the second rotatable knob 80 may comprise tactile elements (refs. 72, 82) configured to reduce a user's cognitive load during loading of the replacement heart valve implant 10 into the implant holding portion 60 and during deployment of the replacement heart valve implant 10 from the implant holding portion 60 of the delivery device 30. In some embodiments, the first tactile element 72 of the first rotatable knob 70 may be configured to reduce a user's cognitive load during loading of the first feature and/or the proximal portion of the replacement heart valve implant 10 into the implant holding portion 60 of the delivery device 30 and during deployment of the first feature and/or the proximal portion of the replacement heart valve implant 10 from the implant holding portion 60 of the delivery device 30. The term “cognitive load” may be understood as the amount of mental effort or working memory resources required to be utilized. A higher cognitive load lowers the percentage of information that is stored in long term memory because it overwhelms the working memory. In some embodiments, the second tactile element 82 of the second rotatable knob 80 may be configured to reduce a user's cognitive load during loading of the second feature and/or the distal portion of the replacement heart valve implant 10 into the implant holding portion 60 of the delivery device 30 and during deployment of the second feature and/or the distal portion of the replacement heart valve implant 10 from the implant holding portion 60 of the delivery device 30. Other configurations are also contemplated. The first tactile element 72 and the second tactile element 82 may reduce the user's cognitive load (e.g., what the user is searching their memory to remember) by using tactile features to communicate the meaning and/or intended use of the first rotatable knob 70 and the second rotatable knob 80, respectively.

In some embodiments, the handle 40 may comprise a landmark ring 90 disposed proximate a distal end of the second rotatable knob 80. The landmark ring 90 may comprise a plurality of distally facing directional markings 92 configured to aid in loading the replacement heart valve implant 10 into the implant holding portion 60 of the delivery device 30. In some embodiments, the landmark ring 90 may be frictionally engaged with the second rotatable knob 80. In some embodiments, the handle 40 may comprise a friction element (not shown) such as an O-ring, a gasket, etc., in engagement with the second rotatable knob 80 and the landmark ring 90. In some embodiments, the friction element may permit relative movement between the second rotatable knob 80 and the landmark ring 90 when the landmark ring 90 is rotated (e.g., the landmark ring 90 may be rotated relative to the second rotatable knob 80, and/or the landmark ring 90 may be rotated without rotating the second rotatable knob 80), such as when calibrating the relative positions of the second rotatable knob 80 and the implant holding portion 60 of the delivery device 30. In some embodiments, the friction element may permit and/or cause the landmark ring 90 to rotate along with the second rotatable knob 80 when the second rotatable knob 80 is rotated relative to the handle 40 (e.g., absent any restrictive or holding force capable of overcoming the relative friction between the second rotatable knob 80 and the landmark ring 90 caused by the friction element). For example, once calibrated, the landmark ring 90 may be a slave to the second rotatable knob 80. In some embodiments, the first rotatable knob 70 may comprise a plurality of distally facing directional markings 78 configured to aid in loading the replacement heart valve implant 10 into the implant holding portion 60 of the delivery device 30.

In some embodiments, the delivery device 30 and/or the handle 40 may comprise a safety pin 48, or another safety feature, configured to engage with the handle 40 and/or elements of the delivery device 30 disposed within the handle 40 to prevent rotation of the second rotatable knob 80 relative to the handle 40. In some embodiments, the safety pin 48 may be configured to engage with the second rotatable knob 80 and the handle 40 to prevent movement and/or rotation of the second rotatable knob 80 relative to the handle 40. In some embodiments, the safety pin 48 may be configured to extend through the second rotatable knob 80 to engage with the handle 40. In some embodiments, the second rotatable knob 80 may comprise an aperture 81 configured to be aligned with an aperture 41 formed in the handle 40, and the safety pin 48 may be inserted into and/or through the aperture of the second rotatable knob 80 and the safety pin 48 may be received by and/or inserted into the aperture 41 of the handle 40. Other configurations are also contemplated. In at least some embodiments, with the safety pin 48 in place (e.g., extending through the second rotatable knob 80), the landmark ring 90 may be rotatable relative to the second rotatable knob 80 once sufficient rotational force is applied to overcome friction between the landmark ring 90 and the second rotatable knob 80 (e.g., to overcome the friction element).

Returning to FIG. 3, in some embodiments, a distal portion of the delivery device 30 and/or the elongate shaft assembly 50 may comprise the implant holding portion 60 configured to engage with and/or constrain the replacement heart valve implant 10 and/or the expandable framework 12 in the radially collapsed configuration. The elongate shaft assembly 50 may include an outer tubular member 52 extending distally from the handle 40 and an inner shaft 54 extending distally from the handle 40 within the outer tubular member 52 to a distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and about and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle 40.

In some embodiments, the handle 40 may be configured to manipulate and/or translate the proximal sheath 62 and/or the distal sheath 64 relative to each other using the first rotatable knob 70 and/or the second rotatable knob 80, as discussed further below. In some embodiments, the handle 40 and/or the second rotatable knob 80 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle 40 and/or the first rotatable knob 70 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 10 to a treatment site (e.g., the native heart valve, the aortic valve, etc.), the replacement heart valve implant 10 may be disposed at least partially within the proximal sheath 62 and/or the distal sheath 64 in the radially collapsed configuration in a closed configuration of the implant holding portion 60. In some embodiments, the proximal sheath 62 and/or the distal sheath 64 may collectively define the implant holding portion 60 of the delivery device 30. In some embodiments, the implant holding portion 60 may be configured to constrain the replacement heart valve implant 10 in the radially collapsed configuration when the implant holding portion 60 is in the closed configuration. In some embodiments, the replacement heart valve implant 10 may be releasably coupled to and/or releasably engaged with the intermediate tubular member 56 and/or a stent holder (not shown) when the replacement heart valve implant 10 is constrained within the implant holding portion 60 of the delivery device 30 in the radially collapsed configuration.

In some embodiments, the proximal sheath 62 may be configured to cover the proximal portion of the replacement heart valve implant 10 in the radially collapsed configuration when the implant holding portion 60 is in the closed configuration, and the distal sheath 64 may be configured to cover the distal portion of the replacement heart valve implant 10 in the radially collapsed configuration when the implant holding portion 60 is in the closed configuration. In some embodiments, the replacement heart valve implant 10 may be constrained in the radially collapsed configuration by the proximal sheath 62 and the distal sheath 64 in the closed configuration of the implant holding portion 60. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10. In some embodiments, the proximal sheath 62 may be axially spaced apart from the distal sheath 64 in the closed configuration by less than 15% of an overall length of the replacement heart valve implant 10. In some embodiments, the proximal sheath 62 may be axially spaced apart from the distal sheath 64 in the closed configuration by less than 10% of an overall length of the replacement heart valve implant 10. In some embodiments, the proximal sheath 62 may be axially spaced apart from the distal sheath 64 in the closed configuration by less than 5% of an overall length of the replacement heart valve implant 10. Other configurations are also contemplated.

In some embodiments, the stent holder (not shown) may be fixedly attached to the elongate shaft assembly 50. In some embodiments, the stent holder may be fixedly attached to the intermediate tubular member 56 of the elongate shaft assembly 50. In some alternative embodiments, the stent holder may be monolithically formed with the elongate shaft assembly 50 and/or the intermediate tubular member 56. In some embodiments, the stent holder may be configured to engage the expandable framework 12 in the radially collapsed configuration and/or when the replacement heart valve implant 10 is constrained within the implant holding portion 60 of the delivery device 30. In some embodiments, the stent holder may include at least one projection configured to engage the expandable framework 12 and/or the lower crown 14 in the radially collapsed configuration.

Returning briefly to FIG. 4, it is noted that the plurality of distally facing directional markings 78 of the first rotatable knob 70 and the plurality of distally facing directional markings 92 of the landmark ring 90 are not used during deployment of the replacement heart valve implant 10. Instead, these markings are used during loading of the replacement heart valve implant 10 into the implant holding portion 60 before the implantation procedure.

In some embodiments, the plurality of distally facing directional markings 78 of the first rotatable knob 70 and/or the plurality of distally facing directional markings 92 of the landmark ring 90 may correspond to relative movements of elements of the delivery device 30 and/or the implant holding portion 60 during loading of the replacement heart valve implant 10 into the implant holding portion 60. For example, the plurality of distally facing directional markings 78 of the first rotatable knob 70 may correspond to movement of the proximal sheath 62 relative to the distal sheath 64 and/or the intermediate tubular member 56, and the plurality of distally facing directional markings 92 of the landmark ring 90 may correspond to movement of the distal sheath 64 relative to the proximal sheath 62 and/or the intermediate tubular member 56. In some embodiments, the plurality of distally facing directional markings 78 of the first rotatable knob 70 and/or the plurality of distally facing directional markings 92 of the landmark ring 90 may correspond to specific start and stop locations of the first rotatable knob 70 and the second rotatable knob 80 associated with various steps of the loading procedure. It may be useful for a user positioned at the implant holding portion 60 to see these markings and/or be privy to this information while loading the replacement heart valve implant 10 into the implant holding portion 60, which may negate a need for the user to move to a position at and/or over the handle 40 in order to see loading and/or calibration markings. Other configurations and/or relationships are also contemplated.

In some alternative configurations, the landmark ring 90 may be fixedly attachable to the second rotatable knob 80, such as via fastener(s) or adhesive(s) applied to and/or inserted into one or more holes 94 (note that the holes themselves are hidden from view in FIGS. 3-4) formed in the landmark ring 90. For example, the relative positions of the implant holding portion 60 and/or elements thereof may be set and/or calibrated during manufacturing and/or assembly of the delivery device 30, and the landmark ring 90, which may aid in setting the proper locations and/or calibrating the relative locations of these features, may be used in only that setting, thereby reducing procedure steps and/or time during implantation of the replacement heart valve implant 10. Other configurations are also contemplated.

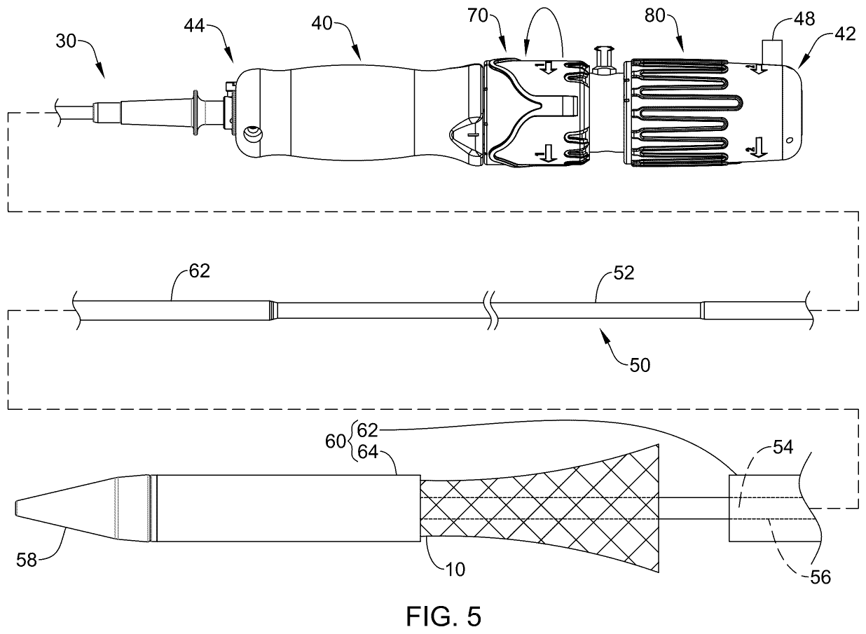

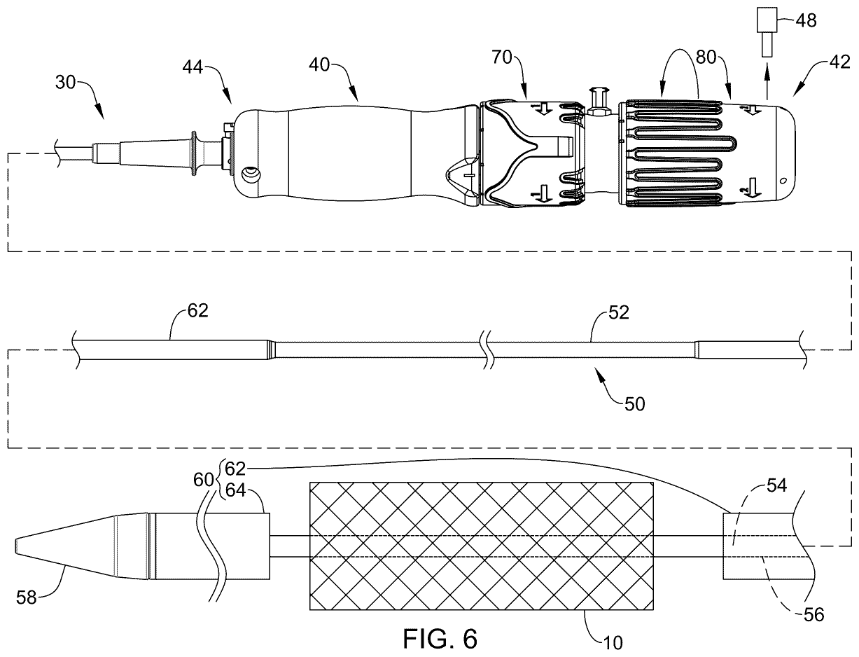

FIGS. 5-6 schematically illustrate selected aspects related to deploying the replacement heart valve implant 10 from the delivery device 30. In use, the delivery device 30 may be advanced percutaneously through the vasculature to a position adjacent to the treatment site (e.g., the native heart valve, the aortic valve, etc.). For example, the delivery device 30 may be advanced through the vasculature and across the aortic arch to a position adjacent to the aortic valve. Alternative approaches to treat a defective aortic valve and/or other heart valve(s) are also contemplated with the delivery device 30. Orientation of the replacement heart valve implant 10 and/or elements thereof, shown schematically in FIGS. 5-6, may be gleaned from FIGS. 1-3.

In some embodiments, positioning the replacement heart valve implant 10 and/or the expandable framework 12 within the native heart valve (e.g., the aortic valve) may be accomplished by locating a primary visual indicator (e.g., a marker band, etc.) relative to the native heart valve (e.g., the aortic valve). During visualization, the native heart valve (e.g., the aortic valve) may be identified and/or visualized under fluoroscopy using known means and/or methods, such as contrast injection. Other configurations are also contemplated.

After positioning the replacement heart valve implant 10 and/or the expandable framework 12 at the treatment site and/or within the native heart valve (e.g., the aortic valve), the first rotatable knob 70 may be rotated relative to the handle 40 to translate the outer tubular member 52 and/or the proximal sheath 62 proximally relative to the distal sheath 64 and/or the intermediate tubular member 56, thereby exposing and/or deploying the first feature and/or the proximal portion of the replacement heart valve implant 10 from the implant holding portion 60 of the delivery device 30, as seen in FIG. 5. As noted herein, rotation of the first rotatable knob 70 relative to the handle 40 may be configured to deploy the first feature and/or the proximal portion of the replacement heart valve implant 10 from the implant holding portion 60 of the delivery device 30. In some embodiments, the first rotatable knob 70 may be rotated counterclockwise, as viewed from the proximal end 42 of the handle 40 to the distal end 44 of the handle 40, to deploy the first feature and/or the proximal portion of the replacement heart valve implant 10 from the implant holding portion 60 of the delivery device 30. In some embodiments, rotation of the first rotatable knob 70 relative to the handle 40 to deploy the first feature and/or the proximal portion of the replacement heart valve implant 10 from the implant holding portion 60 of the delivery device 30 may involve a series of slow, relatively short turns adding up to about 540 degrees of rotation, about 720 degrees of rotation, about 900 degrees of rotation, etc., using a pinch grip. Other configurations are also contemplated.

After exposing and/or deploying the first feature and/or the proximal portion of the replacement heart valve implant 10 from the implant holding portion 60 of the delivery device 30, positioning of the replacement heart valve implant 10 and/or the expandable framework 12 relative to the treatment site (e.g., the native heart valve, the aortic valve, etc.) may be verified using appropriate visualization means. In some embodiments, the replacement heart valve implant 10 may be recaptured and/or repositioned at and/or within the treatment site (e.g., the native heart valve, the aortic valve, etc.) as necessary.

After verifying the positioning of the replacement heart valve implant 10 and/or the expandable framework 12 relative to the treatment site (e.g., the native heart valve, the aortic valve, etc.), and/or concluding that moving forward with the implantation procedure is desired, the safety pin 48 may be disengaged and/or removed from the handle 40 and/or the second rotatable knob 80, as seen in FIG. 6.

Subsequently (e.g., after disengaging and/or removing the safety pin 48), the second rotatable knob 80 may be rotated relative to the handle 40 to translate the inner shaft 54 and/or the distal sheath 64 distally relative to the proximal sheath 62 and/or the intermediate tubular member 56, thereby exposing and/or deploying the second feature and/or the distal portion of the replacement heart valve implant 10 from the implant holding portion 60 of the delivery device 30, as seen in FIG. 6. As noted herein, rotation of the second rotatable knob 80 relative to the handle 40 may be configured to deploy the second feature and/or the distal portion of the replacement heart valve implant 10 from the implant holding portion 60 of the delivery device 30. In some embodiments, the second rotatable knob 80 may be rotated counterclockwise, as viewed from the proximal end 42 of the handle 40 to the distal end 44 of the handle 40, to deploy the second feature and/or the distal portion of the replacement heart valve implant 10 from the implant holding portion 60 of the delivery device 30. In some embodiments, rotation of the second rotatable knob 80 relative to the handle 40 to deploy the second feature and/or the distal portion of the replacement heart valve implant 10 from the implant holding portion 60 of the delivery device 30 may involve a fast, relatively large degree of rotation using a power grip. In some embodiments, the relatively large degree of rotation may be between about 90 degrees and about 120 degrees, between about 95 degrees and about 115 degrees, between about 100 degrees and about 110 degrees, etc. In one non-limiting example, the relatively large degree of rotation may be about 103 degrees. Other configurations are also contemplated.

The first rotatable knob 70 and the second rotatable knob 80 may provide tactile feedback to the user regarding which feature (e.g., the first feature or the second feature) or portion (e.g., the proximal portion of the distal portion) of the replacement heart valve implant 10 and/or the expandable framework 12 is being deployed and/or unsheathed. The first rotatable knob 70 and the second rotatable knob 80 may include features (e.g., the first tactile element 72 and the second tactile element 82, respectively) providing clear and descriptive tactile information related to proximal or distal portions of the procedure. Additionally, in some embodiments, the second axial length 84 of the second rotatable knob 80 being longer and/or greater than the first axial length 74 of the first rotatable knob 70 provides further differentiation and/or better grip for the user. In some embodiments, the first axial length 74 of the first rotatable knob 70 may be configured to facilitate a pinch grip, and the second axial length 84 of the second rotatable knob 80 may be configured to facilitate a power grip. Other configurations and/or variations in axial length of the one or more rotatable knobs are also contemplated. Tactile feedback corresponding to which feature (e.g., the first feature or the second feature) or portion (e.g., the proximal portion of the distal portion) of the replacement heart valve implant 10 and/or the expandable framework 12 is being deployed and/or unsheathed may improve safety and efficiency of the procedure by lowering the probability of rotating the first rotatable knob 70 and the second rotatable knob 80 in an incorrect order, and by providing immediate and clear feedback to the user using only touch. For example, the user may have less (or no) need to look at the handle 40 during the procedure, thereby taking attention away from the visualization means (e.g., a fluoroscopy screen, etc.).

The plurality of distally facing directional markings 78 of the first rotatable knob 70 and/or the plurality of distally facing directional markings 92 of the landmark ring 90 are not easily and/or readily visible to the user during deployment of the replacement heart valve implant 10 because the plurality of distally facing directional markings 78 of the first rotatable knob 70 and/or the plurality of distally facing directional markings 92 of the landmark ring 90 face distally instead of radially outwardly. Therefore, the user positioned over and/or alongside the handle 40 will not have visibility of the plurality of distally facing directional markings 78 of the first rotatable knob 70 and/or the plurality of distally facing directional markings 92 of the landmark ring 90, which may reduce user confusion and/or cognitive load deployment of the replacement heart valve implant 10.

The materials that can be used for the various components of the delivery device and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the delivery device, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A delivery device for a replacement heart valve implant, comprising:
a handle comprising a first rotatable knob and a second rotatable knob; and
an elongate shaft assembly extending distally from the handle, wherein a distal portion of the elongate shaft assembly comprises an implant holding portion configured to constrain the replacement heart valve implant in a radially collapsed configuration;
wherein the replacement heart valve implant comprises a first feature and a second feature;
wherein the first rotatable knob comprises a first tactile element representative of the first feature;
wherein the second rotatable knob comprises a second tactile element representative of the second feature.

2. The delivery device of claim 1, wherein the first tactile element has a first shape corresponding to a shape of the first feature.

3. The delivery device of claim 1, wherein the second tactile element has a second shape corresponding to a shape of the second feature.

4. The delivery device of claim 1, wherein rotation of the first rotatable knob relative to the handle is configured to deploy the first feature from the implant holding portion.

5. The delivery device of claim 1, wherein rotation of the second rotatable knob relative to the handle is configured to deploy the second feature from the implant holding portion.

6. The delivery device of claim 1, wherein the first rotatable knob has a first axial length, and the second rotatable knob has a second axial length greater than the first axial length.

7. The delivery device of claim 1, wherein the first rotatable knob is formed from a different material than the handle.

8. The delivery device of claim 1, wherein the second rotatable knob is formed from a different material than the handle.

9. The delivery device of claim 1, wherein the first rotatable knob comprises an outer surface and the first tactile element extends radially outward of the outer surface of the first rotatable knob.

10. The delivery device of claim 1, wherein the second rotatable knob comprises an outer surface and the second tactile element extends radially outward of the outer surface of the second rotatable knob.

11. The delivery device of claim 1, wherein the first tactile element is monolithically formed with the first rotatable knob.

12. The delivery device of claim 1, wherein the second tactile element is monolithically formed with the second rotatable knob.

13. A delivery device for a replacement heart valve implant, comprising:
a handle comprising a first rotatable knob and a second rotatable knob; and
an elongate shaft assembly extending distally from the handle, wherein a distal portion of the elongate shaft assembly comprises an implant holding portion configured to constrain the replacement heart valve implant in a radially collapsed configuration;
wherein the replacement heart valve implant comprises a first feature and a second feature;
wherein a first tactile element representative of the first feature is embossed on the first rotatable knob;
wherein a second tactile element representative of the second feature is embossed on the second rotatable knob.

14. The delivery device of claim 13, wherein the handle comprises an ergonomic outer surface extending from the first rotatable knob to a distal end of the handle.

15. The delivery device of claim 13, further comprising a safety pin configured to engage with the handle to prevent rotation of the second rotatable knob relative to the handle.

16. The delivery device of claim 15, wherein the safety pin is configured to extend through the second rotatable knob to engage with the handle.

17. The delivery device of claim 13, wherein the handle comprises a landmark ring disposed proximate a distal end of the second rotatable knob, the landmark ring comprising a plurality of distally facing directional markings configured to aid in loading the replacement heart valve implant into the implant holding portion.

18. The delivery device of claim 17, wherein the first rotatable knob comprises a plurality of distally facing directional markings configured to aid in loading the replacement heart valve implant into the implant holding portion.

19. The delivery device of claim 17, wherein the landmark ring is fixedly attachable to the second rotatable knob.

20. A delivery device for a replacement heart valve implant, comprising:
a handle comprising a first rotatable knob and a second rotatable knob; and
an elongate shaft assembly extending distally from the handle, wherein a distal portion of the elongate shaft assembly comprises an implant holding portion configured to constrain the replacement heart valve implant in a radially collapsed configuration;
wherein the first rotatable knob and the second rotatable knob comprise tactile elements configured to reduce a user's cognitive load during deployment of the replacement heart valve implant from the implant holding portion.
